(19) United States
(12) Patent Application Publication
(10) Pub. No.: US 2005/0117118 A1
(43) Pub. Date: Jun. 2, 2005

(54) Digital ophthalmic workstation

(76) Inventors: David Miller, Brookline, MA (US); Ernesto E. Blanco, Belmont, MA (US); Steve K. Burns, Durham, NH (US); Robert Stegmann, Lynnwood Glen (ZA); Ramgopal Rao, Irvine, CA (US)

Correspondence Address: BROMBERG & SUNSTEIN LLP, 125 SUMMER STREET, BOSTON, MA, 02110-1618, US

(21) Appl. No.: 10/969,226

(22) Filed: Oct. 20, 2004

Related U.S. Application Data
(63) Continuation-in-part of application No. 10/265,303, filed on Oct. 4, 2002.
(60) Provisional application No. 60/327,323, filed on Oct. 5, 2001.
(60) Provisional application No. 60/348,545, filed on Jan. 16, 2002.

(51) Int. Cl. A61B 18/18; G09G 5/00; A61B 3/00

(52) U.S. Cl. 351/246; 606/4

(57) ABSTRACT

A method of providing visual documentation and information for a surgical procedure. The method includes, providing at least one video camera for generating a video signal in a digital format, capturing an image of an eye and displaying the image on a monitor. A first template is created having a graphical content pertinent to the eye. The first template is displayed such that the first template is overlaid contemporaneously with the image of the eye on a display monitor.

This application is a continuation-in-part application of a U.S. patent application entitled “A System and Method of Providing Visual Documentation during Surgery,” Ser. No. 10/265,303, filed Oct. 4, 2002. This application also claims priority from both U.S. provisional patent application Ser. No. 60/327,323, filed Oct. 5, 2001, entitled “An Intelligent Ophthalmic Microsurgical System” and U.S. provisional patent application Ser. No. 60/348,545, filed Jan. 16, 2002, entitled “A Failsafe, Multiview, Electro-optical, Cluster for Ophthalmic Microsurgery.” Each of the patent applications described in this paragraph is hereby incorporated by reference in its entirety.

### TECHNICAL FIELD

The present invention relates to a method and device for providing computer-generated aids for ophthalmic surgery, and more particularly, for visualization of ophthalmic structures.

### BACKGROUND ART

During an ophthalmic surgical procedure using a standard operating microscope, a surgeon may need to make various measurements, such as incision length, the size of an anatomic structure, or distance between anatomic structures. Such measurements are either done by superimposing a reticle in the eyepiece over the area under consideration, or by using a ruler and making a direct measurement in the field of operation. In both cases, the operator often must estimate if the edge of the structure measured falls between the marks on the ruler or on the reticle.

Additionally, during the ophthalmic surgical procedure, the surgeon often marks areas to be excised or sutured with an inked marker. During the operation, the ink tends to be washed away because of frequent irrigations necessary to keep the surface of the eye moist, making such excisions or sutures difficult.

Furthermore, either when operating alone or with an assistant, a surgeon may need advice pertaining to the surgical procedure, such as when performing a new or complicated operation or when facing an unexpected complication. It is important that such advice be provided in a timely and reliable manner. Advisors in remote locations may not be able to observe the surgical subject, making it difficult to provide advice. Additionally, the level of expertise available may not be able to provide proper guidance.

### SUMMARY OF THE INVENTION

In an embodiment of the present invention, a method of providing visual documentation for an ophthalmic surgical procedure is provided. The method includes providing at least one video camera, at least one monitor and a processor. An image of an eye is captured using the video camera. A template having a graphical content that is useful to the surgeon performing a surgical procedure is displayed contemporaneously with the image of the eye on the monitor.

In some embodiments of the invention, the captured image is a stereoscopic view pair and the image of the eye displayed on the monitor is a stereoscopic image. In other embodiments, the image is a single view image.

In a further embodiment of the invention, the image of the eye is captured such that reflections from structures in the eye are reduced. In one embodiment, the eye is illuminated from a given angle and the video camera that captures the image is positioned and aimed to minimize the reflected light captured by the camera. In other embodiments, illuminating light is sequenced from a series of angles and video cameras capture a sequence of images. The sequence of images is then processed to form a composite image with reduced reflections.

In other embodiments of the invention, a system that can respond to a wide dynamic range of illumination is provided to reduce the saturation of highlights and thereby to reduce the effects of glare. In some embodiments of the invention, the wide dynamic range is achieved using a video camera with adjustable sensitivity and using corresponding scaling techniques. In other embodiments, the wide dynamic range is achieved by illuminating the first eye with a variable brightness light source. The brightness of the light source may be adjusted by determining the illumination level at specified points in the captured image and then adjusting the illumination level. In some embodiments of the invention, this adjustment may be made with a feedback loop.

In various embodiments of the invention, the captured image may be processed to change the resolution, contrast, brightness, magnification and color of the image. The image may also be processed to enhance an edge in the image. In some embodiments of the invention, a portion of the image of the eye may be magnified at higher magnification level than another portion of the eye. The portion of the eye with the higher magnification level is shown in greater detail, while the rest of the image provides awareness to a surgeon of the situation outside of the highly magnified portion. In other embodiments of the invention, the eye may be illuminated with laser light. An opaque screen is provided to shield operating room personnel from exposure to the light and the surgeon may view the image of the eye on the display monitor, rather than directly. Thus, danger to sensitive tissues of operating room personnel may be reduced.

In other embodiments of the invention, the eye may be selectively illuminated with specified wavelengths or ranges of wavelengths of light. Further, the video camera that captures the image may have a variable response to light according to the wavelength. Thus, the image displayed to the surgeon on the monitor may emphasize certain wavelengths of light over others to highlight particular structures of the eye.

In other embodiments of the invention, a method is provided to measure distances on the eye. Two points in the image of the eye are identified: in some embodiments the points are identified on the display monitor using a pointing device. The distance between the points is then displayed on the monitor. In another embodiment of the invention, the template shows the major axes of astigmatism of the cornea and the optical center of the cornea. As a procedure progresses, these measurements may be updated and redisplayed for the surgeon.

In some embodiments of the invention, a template is displayed on the image of the eye on the monitor that show the position and extent of an incision to guide a surgeon. The incision may be for one of cataract removal and corneal repair. In other embodiments of the invention, the templates shows a suturing pattern. In other embodiments of the invention, the template includes an indication of the position and orientation for a surgical instrument during a procedure.

In other embodiments of the invention, a template may provide guidance for placing an implant, including a transplant, in an eye. The template may identify the placement and shape of the implant, so that the surgeon may make an appropriately sized incision at the site. Further, once a site has been prepared for an implant or a transplant, the surgeon may identify with an input device the size and shape of the prepared site. A template may then be generated by the processor to match the outline of the prepared site. This template may be superimposed on the image of the eye at the site where the transplant tissue is to be harvested, as a guide to the surgeon.

In other related embodiments of the invention, a template may include surgical advice pertinent to the surgical subject. Such advice may be interpretive. Surgical advice may include, for example, providing pattern recognition. The template may include a selectable suturing pattern into the template. Displaying the template overlaid contemporaneously with the image of the eye may include displaying a plurality of display windows on one or more displays. Audio pertinent to the surgical subject may be provided. In other embodiments of the invention, data may be received and displayed on the display monitor. The data may include data characterizing the status of a patient, or the status of equipment, or both. The data may be updated in real-time and may be displayed on a display surface that shows the image of the eye.

### BRIEF DESCRIPTION OF THE DRAWINGS

The foregoing features of the invention will be more readily understood by reference to the following detailed description, taken with reference to the accompanying drawings, in which:

FIG. 1 is a block diagram of a stereoscopic surgical microscopic system 100, in accordance with one embodiment of the invention;

FIG. 2 is a pictorial view of an operating table equipped with a stereoscopic surgical microscopic system, in accordance with one embodiment of the invention;

FIG. 3 is a cross-sectional view of a slit lamp used in conjunction with a stereoscopic video camera, in accordance with one embodiment of the invention;

FIG. 4 shows a cross-sectional view of an ophthalmoscope used in conjunction with a stereoscopic video camera, in accordance with one embodiment of the invention; and

FIG. 5 shows a sample template overlaid on an image of an eye, in accordance with one embodiment of the invention;

FIG. 6 illustrates display of an image of an eye that includes selective magnification of the image according to an embodiment of the invention;

FIG. 7 shows the relationship of lights sources and video cameras for capturing images for stereoscopic viewing according to an embodiment of the invention;

FIG. 8 shows the positioning of light source and camera to reduce light reflection artifacts in capturing an image of an eye; and

FIG. 9A-9B shows a template overlaid on the image of an eye to aid positioning of a surgical instrument.

### DETAILED DESCRIPTION OF SPECIFIC EMBODIMENTS

In various embodiments of the invention, a method is provided for visualization of the structures of the eye. Video processing and digital image processing techniques enhance the image improving an ophthalmologist's ability to see the structures clearly. In specific embodiments of the invention, a computer is employed to process the captured eye images and to overlay information that is useful for the ophthalmologist for performing therapeutic procedures on the eye.

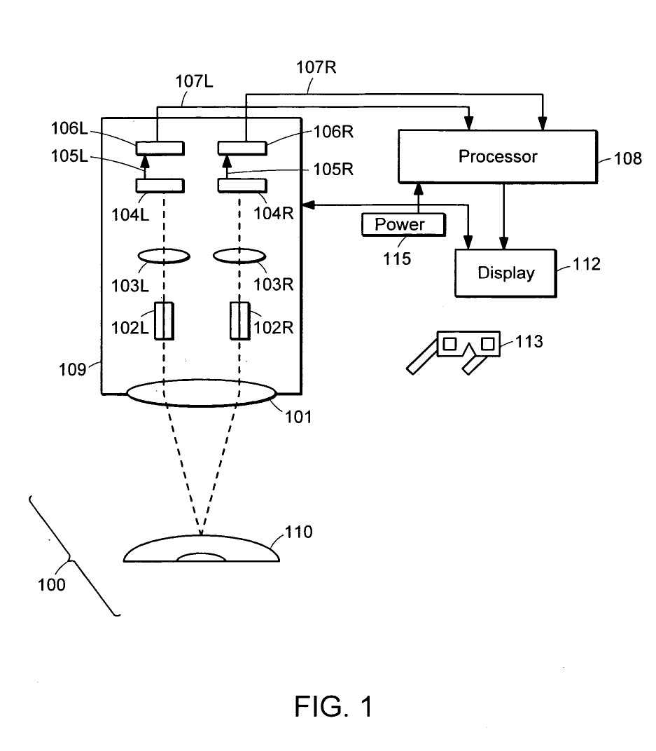

FIG. 1 is a block diagram of a stereoscopic surgical microscopic system 100, in accordance with one embodiment of the invention. Such a system may be used in, but not limited to, ophthalmic surgery, cerebral surgery, cosmetic surgery, and ear, nose and throat surgery. The system includes at least one stereoscopic video camera 109 for generating a video signal(s) representing a stereoscopic view pair of a surgical subject 110. The video signal is provided to one or more displays 112. An intelligence component, which may include, without limitation, a processor 108, provides a template having a graphical content pertinent to the subject of the stereoscopic view pair. The template is displayed on at least one of the displays 112, and may be advantageously overlaid contemporaneously with the stereoscopic view pair.

Stereoscopic video camera 109 may include, without limitation, a beam splitter 101 that splits an optical image of the surgical subject 110 into left and right images representing the stereoscopic view pair of subject 110. The left and right images are magnified by magnification-varying optical units 102L and 102R, and focused by lenses 103L and 103R onto imaging devices 104L and 104R, respectively. Imaging devices 104L and 104R, which may include, for example, a Charge-Coupled Device (CCD), convert the left and right optical images into electrical signals 105L and 105R, respectively. Electrical signal 105L is provided to image processor 106L which outputs a video signal 107L representing the left image of the subject, while electrical signal 105R is provided to image processor 106R which outputs a video signal 107R representing the right image of the subject 110. Note that in various embodiments, the stereoscopic camera may include, instead of a beam splitter, two separate cameras that are used to capture the image from two different angles.

The video signals 107L and 107R from stereoscopic camera are fed into a processor 108, which, in part, may perform image signal processing and/or format the signals 107L and 107R so as to drive the one or more displays 112. Processor 108 may include, without limitation, one or more microprocessors, programmable logic arrays, and/or other logic circuits, as known in the art.

Numerous methodologies known in the art may be used in displaying the three dimensional image. For example, and without limitation, a Liquid Crystal Display (LCD) may be placed over a display screen. The LCD produces fast alternating polarization that is synchronized with alternating presentation of the left and right images on the display screen. Also known are systems in which the left and right images are displayed simultaneously, with each image being polarized in a different configuration. For example, the left and right images may be displayed side-by-side.

Typically, a viewer of the display(s) 112, who may be a surgeon, an assistant surgeon, a nurse, an anesthesiologist and/or any other observer, wears special spectacles 113 so that the left eye receives the left image and the right eye receives the right image. The spectacles may be one of many types as known in the art, such as, but not limited to, special polarizing lenses or alternating occlusion lenses. Upon receiving the two images (i.e. the stereoscopic view pair), the viewer's brain triangulates the left and right images, as seen with the left and right eyes, respectively, such that the viewer perceives a three-dimensional image.

In other embodiments of the invention, a non-stereoscopic (i.e., monocular) view may be captured by a video camera and presented for viewing to a surgeon. A system similar to the system shown in FIG. 1 is provided, except that the image captured by the camera and the video signal generated represents a monocular view of a surgical subject 110, such as an eye. The video signal is provided to one or more displays 112. An intelligence component, which may include, without limitation, a processor 108, provides a template having a graphical content pertinent to the subject of the view, e.g., an eye. The template is displayed on at least one of the displays 112, and may be advantageously overlaid contemporaneously with the image of the surgical subject. In such a system, special glasses 113 or other viewing devices to separate stereoscopic view pairs for viewing are not required. In other embodiments of the invention, a mix of stereoscopic and monocular images are captured and presented on one or more display monitors. These images may be presented in any combination on a single display 112 or on multiple displays 112. In the various embodiments of the invention described below, embodiments described in terms of a stereoscopic viewing system may also be created using a monocular viewing system or any mixture of the two techniques. All such embodiments are intended to be within the scope of the invention, as defined by the appended claims.

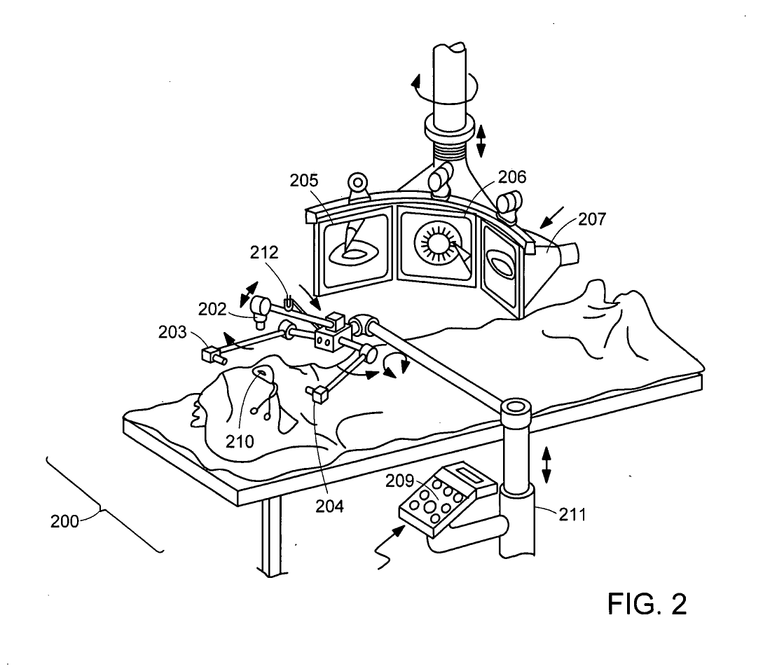

FIG. 2 shows a pictorial view of an operating table equipped with a stereoscopic surgical microscopic system 200, in accordance with one embodiment of the invention. The system includes at least one stereoscopic video camera 202-204. Advantageously, at least two stereoscopic video cameras 202-204 may be provided so that a backup camera is readily available if a camera fails. Each camera 202-204 in the system 200 can be aimed to view the subject 210 from two different directions so as to display different perspectives of the subject 210. This can be particularly relevant in eye surgery, since certain transparent structures of the eye can only be seen if illuminated at specific angles.

Each camera 202-204 may be removably mounted to a boom 211. Boom 211 may include, without limitation, a stand that is adjustable in height, and/or one or more arms that may move in an arbitrary direction. Thus, each camera's position can be properly adjusted so as to provide a desired view of a subject 210. In other embodiments of the invention, the camera(s) may be attached to a headpiece worn by a patient, so as to be focused on, for example, an eye upon which surgery is to be conducted. Thus, the camera(s) will remain focused on the eye regardless of any movement by the patient.

The images from each camera may be selectably displayed on one or more displays 205-207. Each display(s) 205-207 may have a high resolution of, for example, 1280 pixels×1024 pixels or greater. Display(s) 205-207 may be, without limitation, a monitor or a flat-panel display. Multiple images may be displayed on a single display, using, for example, a multi-window graphical operating system.

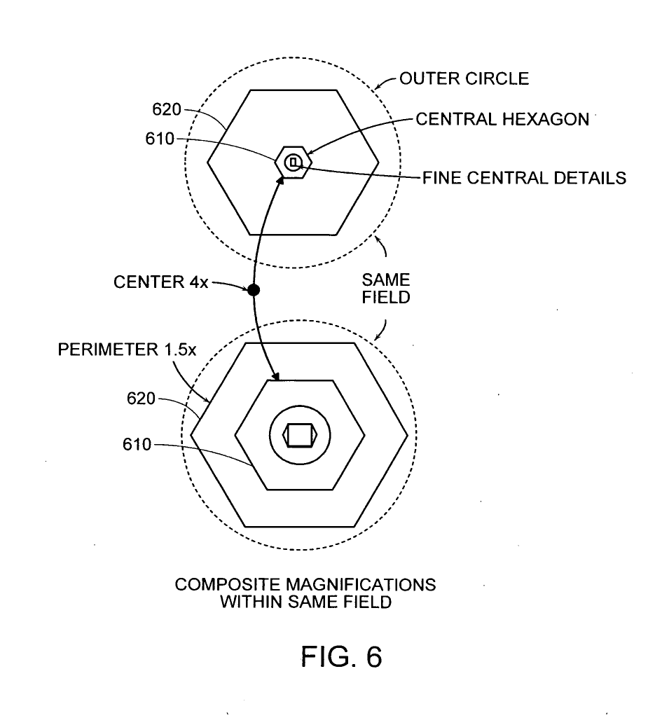

Through the use of video displays 205-207, the system 200 advantageously provides a larger field of view compared to a traditional operating microscope. With the traditional operating microscope, the size of the viewable field is inversely proportional to the level of magnification. Thus at higher magnifications, the size of the field or view is small. With video system 200, magnification is augmented by the size of the video display(s) 205-207. The optical magnification can be arranged to allow a larger visual field, and then extra magnification of the objects in the field is supplied by a larger display area. The larger display field allows the surgeon to notice potential problems at the periphery of the operative field, an area that may not be seen with a conventional microscope using high magnification. In some embodiments of the invention, as shown in FIG. 6, different levels of magnification maybe applied to different portions of an image. For example, the center portion 610 of an image of an eye may be displayed at a higher magnification level than adjacent portions of the image 620. This technique allows fine detail to be presented for one portion of the eye while presenting a lesser level of detail for the rest of the eye. Thus, a surgeon may maintain awareness of the impact of his or her work on the entire eye, while concentrating on a particular eye structure. Selective magnification is not limited to a single region of the image or the central portion of the image but may be applied to any region or set of regions within the image. The surgeon may use a pointing device or other input device to identify the region or regions in which the magnification is to be changed.

System 200 may include an operator interface 209. Operator interface 209 provides the surgeon and/or other observer (referred to hereinafter as the “surgeon”) the capability to control various aspects of system 200. For example and without limitation, the surgeon may control, via the operator interface 209, zoom functionality, focusing of cameras 202-204, and/or which camera image or other graphical content is displayed on a particular display. Graphical content on each display may be controlled by software driven menus or taskbars using point and click methodology or other similar means known in the art. Operator interface 209 may include, without limitation, a keyboard, a trackball, a joystick, and/or a mouse. Operator interface may also include a remote control, a foot control that frees the surgeon's hands, and/or one or more memory devices, such as a semiconductor, magnetic, optical or other memory device.

The system 200 includes at least one illumination source 212. The illumination source 212 may be, without limitation, an incandescent bulb (e.g., a xenon arc bulb) that is typically used with traditional operating microscopes. However, incandescent bulbs are subject to burn-out and may need replacement during surgery. Instead of an incandescent bulb, a light-emitting diode (LED) may advantageously be utilized, in accordance with one embodiment of the invention. LEDs typically have a higher reliability than incandescent bulbs, and may function for thousands of hours before failure. Additionally, the reliability of the system can further be improved if a second illumination source is provided, which may be attached to an auxiliary camera. In certain surgeries, an eye may be illuminated by a laser, such as for laser vision correction. In such surgeries, an opaque barrier may be placed between the laser light and operating room personnel, including the surgeon. The surgeon may view the image of the eye on the display monitor, rather than viewing the eye directly. Thus, dangerous reflections of laser light from the eye or other surfaces to sensitive tissues of operating room personnel may be reduced.

The illumination source 212 provides enough light to produce a stereoscopic video image. This single image may then be provided and viewed simultaneously on a plurality of displays. Thus, compared to traditional microscopic systems, which typically require an optical beam splitter for each observer, with each beam splitter requiring additional illumination, less illumination is required. This is particularly important in eye surgery. For example, the retina of a patient can be damaged by high light intensity. Note that in various embodiments, the video image generated by the system 200 can be electronically brightened.

Since the illumination used in the system 200 for multiple observers is typically less than that used in traditional microscopic, as described above, and below the level of illumination that can cause damage to the eye, the illumination in the system 200 may be advantageously increased. For example, illumination may be increased (yet remain within safe limits), such that a polarization filter can be placed over the camera lens. The polarization filter can eliminate annoying reflections that can create glare and obscure key anatomic features during surgery.

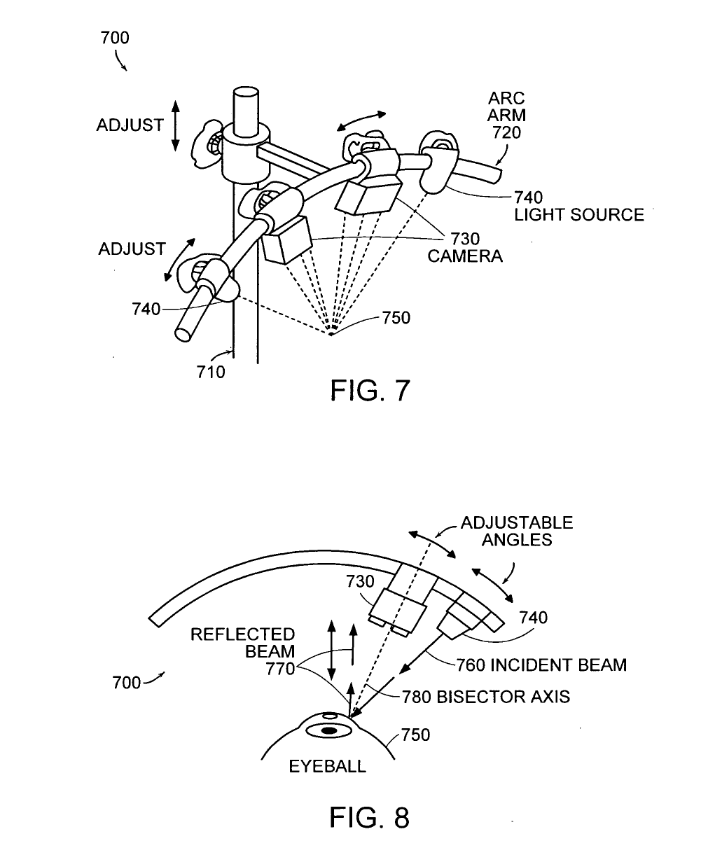

In another embodiment of the invention, other methods of reducing reflections in the displayed image of an eye are provided. FIG. 7 shows a perspective view of an apparatus 700 to hold and position cameras and light sources. A boom 710 with a curved supporting rod 720 that holds video cameras 730 and light sources 740 may be provided. The positions of the cameras and light sources on the supporting rod and the orientation of these cameras and light sources may be adjusted to determine the angles at which the cameras and light sources are aimed at the subject eye 750. FIG. 8 shows a view of a vertical plane that contains the cameras, light sources and the subject eye. The cameras and light sources may be positioned and aimed to reduce or eliminate reflections in the image captured by the camera. Thus, the surgeon viewing the display monitor sees an image with fewer reflection artifacts. For example, as shown in FIG. 8, the reflected beam 770 from light source 740 will be such that the angle of incidence 760 of the light is equal to the angle of reflection of the light (specular reflection). Thus, if the camera is aimed such that the image is captured along a ray 780 that bisects the incident and reflected rays from the light source, the reflected light in the image may be substantially reduced. In other embodiments of the invention, multiple light sources and multiple cameras are used to capture an image of the eye. The lights and cameras are arranged in an arc around the subject eye with specified angles with respect to the eye and the lights are sequenced on and off. The processor is used to select images or portions of the images of the eye and to form a composite image that reduces reflections. This image is then presented on the display 205-207.

In a further embodiment of the display, a method is provided for reducing the effects of glare in an image of an eye. The image is captured with a system with a wide dynamic range so that the saturation of highlights in portions of the image may be reduced. The wide dynamic range may be achieved, in some embodiments, by using a video camera with adjustable sensitivity, taking a sequence of images of the eye, and using scaling techniques for the various portions of the eye to form a composite image of the eye, thereby reducing the saturation of highlights. In some embodiments of the invention, the wide dynamic range is achieved by illuminating the eye with at least one variable brightness light source and taking a sequence of images of the eye under varying brightness levels. The sequence of the captured images is then processed to form a composite image, reducing the saturation of highlights. The brightness of light for a particular image in the sequence of images may be determined using a feedback loop.

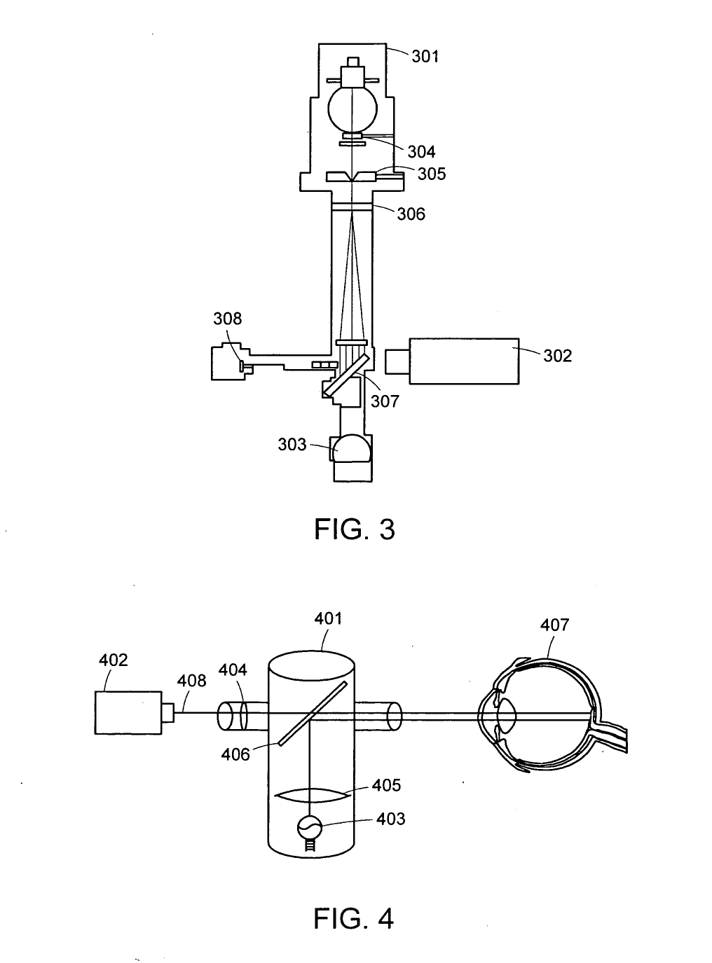

The illumination source 212 may also be a slit lamp or an ophthalmoscope, with the microscope typically used in such systems replaced by a stereoscopic video camera. FIG. 3 is a cross-sectional view of a slit lamp 301 used in conjunction with a stereoscopic video camera 302, in accordance with one embodiment of the invention. The slit lamp 301 is used to enhance viewing of many components of the eye 308 that are almost transparent, such as the lens, cornea, aqueous humor, and vitreous body. The slit lamp 301 does this by maximizing the back scattering of light from these various eye components. Typical components of a slit lamp include, for example, a light illumination source 303, a condensing lens 304, a slit 305, a filter tray 306, and a two way mirror 307. An optical output 408 from slit lamp 301 is input into the stereoscopic video camera 402. In various embodiments, the illumination may be enlarged from a slit 305 to a disk to allow an overall view of the eye 308 as well as to allow photography of the eye 308.

FIG. 4 shows a cross-sectional view of an ophthalmoscope 401 used in conjunction with a stereoscopic video camera 402, in accordance with one embodiment of the invention. Using an ophthalmoscope, internal structures of the eye 407 may be seen, such as the retina, and the surgeon is able to diagnose various eye disorders and some general conditions, such as diabetes and high blood pressure. The ophthalmoscope 401 provides an optical signal 408 that is input into the stereoscopic video camera 402. Typical components of the ophthalmoscope include, for example, a light illumination source 403, two focusing lens 404 and 405, and a two way mirror 406.

The bright illumination from a light source, such as slit lamp or ophthalmoscope, can be annoying to a patient. With the three dimensional video system of the present invention, the video signal representing the stereoscopic view pair may advantageously be stored in memory and analyzed at a later time, so as to minimize the amount of time the eye is illuminated.

In embodiments of the invention, an eye may be illuminated at a selected wavelength or a range of wavelengths. Particular structures in the eye absorb or reflect light more strongly at certain wavelengths. For example, blood vessels just below the surface of the skin, such as the vessels in an eyelid, can be made visible using blue/green light since these wavelengths are strongly absorbed by hemoglobin. Other wavelengths (often in near ultraviolet or near infrared) can make tissue or certain in vivo dyes fluoresce. The illuminating light may be restricted to a wavelength or a range of wavelengths by, for example, applying filters to the illuminating source. Further, the image presented to the display 112 may be filtered, either at the video camera, or other sensor, or electronically. Such filtering can restrict the image viewed to certain wavelengths of light.

Referring back to FIG. 1, to power various components of the system 100 a power source 115 is provided. The power source 115 may be, without limitation, wall current. In other embodiments, one or more system 100 components may be advantageously powered by batteries, which allow continued use of the system in the event of a power outage. The batteries may be rechargeable.

Processor 8 may perform image enhancement on the stereoscopic view pair prior to providing the stereoscopic view pair to the display(s) 112. Image enhancement may include, without limitation, contrast enhancement, enhancement of edges, zoom capability, electronic brightness control and/or providing special coloring to bring out features such that are difficult to detect with conventional microscopy, such as cataract fibers during cataract removal. Image stabilization may be provided, so as to present a stable image regardless of whether the subject is vibrating or otherwise moving.

In various embodiments of the invention, processor 108 may provide a template that has a graphical content pertinent to the subject of the stereoscopic view pair. The template may be displayed such that the template is overlaid contemporaneously with the stereoscopic view pair. For example, the template may be superposed on, and/or juxtaposed next to, the displayed stereoscopic view pair. All or portions of the template may be displayed so as to be perceived by the viewer in three dimensions. Template may be displayed on display(s) 112 in one or more window(s) in combination with, or separate from, the stereoscopic view pair. For example, and without limitation, the template may be displayed using a split screen and/or picture in picture approach.

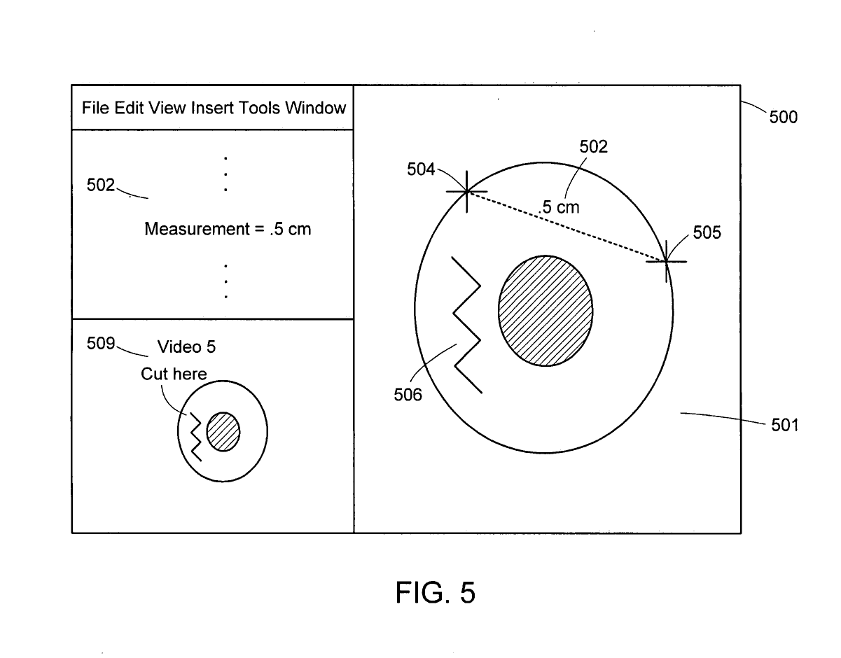

A sample template 500 that incorporates the stereoscopic view pair 501 is shown in FIG. 5 (shown as a two dimensional view for illustrative purposes), in accordance with one embodiment of the invention. The template 500 may provide, without limitation, a measurement(s) 502 related to the displayed stereoscopic view pair 501. For example, the measurement 502 may include an incision length, a size of an anatomical structure, a distance between two anatomical structures, or a position of a structure on a grid superposed on the stereoscopic view. For example, pupil size, the size of a lesion, and a corneal curvature may be measured. Measurements may accurately be calculated by the processor 8 (see FIG. 1) using various methodologies known in the art. For example, the measurement may be calculated based, at least in part, on the number of pixels between two points to be measured (which can then be multiplied, for example, by a predetermined pixel dimension).

In accordance with one embodiment of the invention, the viewer determines the measurement to be made using at least one cursor 504 and 505 that is overlaid on the displayed stereoscopic view 501. The cursor(s) 504 and 505 may be positioned, selected, and/or otherwise controlled using, for example, the operator interface 209 (see FIG. 2) described above. Processor 8 may overlay the cursor(s) 504 and 505 such that the cursor 504 and 505 is viewed in a stereoscopic manner, as known in the art. Measurements provided by the processor 8 may be stored in memory, providing instant recall and protection against memory loss.

The template 500 may provide changes in elevation pertaining to the displayed stereoscopic view pair. Processor 8 may include, for example, a stereogrammetry program, for measuring the elevation changes. This feature may be used, for example and without limitation, at the close of many ophthalmic operations wherein, after all the sutures have been tightened, sterile fluid is injected into the eye to restore normal intra ocular pressure. If too much fluid is injected into the eye, an abnormally high intra ocular pressure will result, leading to complications. Using the stereogrammetry program, the elevation of the edges of the sutured incision can be followed, allowing any incision gaps to be detected. These gaps indicate that intra ocular pressure is too high and accordingly, some saline should be removed.

In accordance with another embodiment of the invention, the template 500 may provide one or more suturing patterns 506 that can be superposed onto the displayed stereoscopic view pair. The suturing patterns 506 may be stored in memory, and may be operator selectable from a software menu using, for example, operator interface 209 described above. Once selected, the suturing pattern 506 may be superposed on the displayed stereoscopic view 501 and, if desired, further modified. Suturing patterns 506 selected by the viewer may be scalable and may be capable of orientation in any direction. In various embodiments, the viewer may be provided the capability to create new suturing patterns from scratch, which may then be saved into memory. Suturing patterns 506 may be displayed, without limitation, in various colors and/or as a wire frame image, such that the viewer can have a clear view of the subject being worked on. In various embodiments, patterns or markings other than suturing patterns may be superposed onto the displayed stereoscopic view pair, as desired. Markings may be, without limitation, alphanumeric, and/or geometric, such as lines, arrows, and circles, and may be used, for example, to label points of interest.

In other embodiments of the invention, the template 500 may show the position and extent of an incision to be made in a surgical procedure. Such types of incisions may include, for example, incisions for cataract removal and for repair of a cornea. A template may show the correct size and position for a capsularhexis procedure, for example, by superimposing a circle on the center of a cataractous lens. Since the capsularhexis is typically 5-6 mm in diameter, templates including circle of 5 mm, 5.5 mm and 6 mm are provided. In another embodiment, a template showing a triangle is provided for assisting a surgeon in removing a portion of the iris during an iridectomy. The size of the base of the triangle is approximately 0.5 to 2.0 mm with the base positoned at the iris edge. In a further embodiment of the invention, the template identifies retinal tissue for dissection.

Other templates that may be displayed show the major axes of astigmatism of the cornea and the optical center of the cornea. In a specific embodiment of the invention, a second template may be overlaid on the eye, showing the major axes of astigmatism of the cornea and the optical center of the cornea as the procedure progresses. The axes of astigmatism and center of the cornea may be determined, for example, by pattern recognition by the processor.

In other embodiments of the invention, the template 500 may provide guidance for placing an implant, including a transplant, in an eye. The template may, for example, identify the placement and shape of the implant, so that the surgeon may make an appropriately sized incision at the site. Further, once a site has been prepared for an implant or a transplant, the surgeon may identify with an input device the size and shape of the prepared site. A template may then be generated by the processor to match the outline of the prepared site. This template may be superimposed on the image of the eye at the exact site where the transplant tissue is to be harvested, as a guide to the surgeon. This technique will be useful, for example, in pterygium surgery where, after the removal of the pterygium, healthy conjunctival tissue is harvested and then sutured in place to cover the site. This technique will also be valuable, for example, for identifying a corneal transplant from a donor eye and, in particular, for displaying a further template that shows the suture pattern for the transplant.

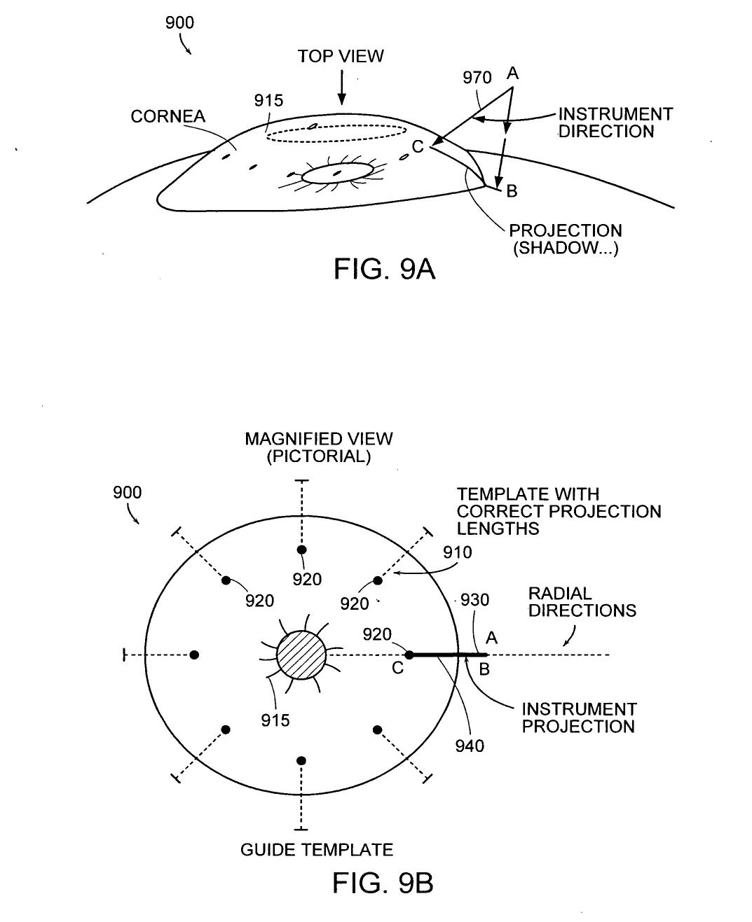

In another embodiment of the invention, the template 500 may indicate the position and angle at which an instrument should be held during a part of a procedure. In certain surgical maneuvers, the angle that the surgeon holds an instrument, such as a scalpel, scissors or a heating probe, is crucial in creating a precise and reproducible effect. FIG. 9B shows a image of an eye on a monitor in a top view 900. A guiding template 910 may be superimposed on the image of the eye 915 on a monitor 112. The template is calculated by projecting the instrument 970, when held at the correct angle, on the image of the eye. The template indicates the position of the tip of an instrument (inner circle of points) 920 and the position of the end of a projection shadow 930 if the instrument is held at the proper angle. Thus, the surgeon is guided by the image of the shadow 940 of his instrument as shown on the monitor, changing the angle of the instrument until the projection shadow 940 matches the marks 920, 930 in the superimposed template. FIG. 9A shows a perspective view of the geometry of the instrument 970 with respect to the projection shadow 940.

In other embodiments of the invention, a variety of data related to a surgical procedure may be received by the system and displayed on the monitor 112 contemporaneously with display of images of eye structures. Such data may include data that is updated periodically and then displayed. Display of such real-time data may be on the same monitor as is used to display other information, such as, without limitation, images of an eye, measurements and instructions. The display of such data may also be in a window on the display surface. Thus, in some embodiments of the invention, the surgeon's attention may be directed to a single display surface or a small number of display surfaces for information, rather than being directed to multiple displays, gauges, and meters in various parts of the operating room. The data received by the system may be received through a variety of electronic interfaces, as are known in the art, and the data received may be manipulated by the processor 108 and may also be stored before the data are presented on a display surface. Data that may be received by the processor and displayed includes, without limitation, data related to a patient, such as a temperature, a refractive power, a wave front map, a topography of an eye structure, an intraocular pressure, an area of a tissue, a volume of a tissue, and a corneal thickness. Other data such as a blood pressure, a pulse, and an oxygen level may be received by the system and displayed. Additional data such as time of day, running time of a procedure or a portion of a procedure, or data describing parameters of ancillary equipments such as pumps and suction devices may be displayed. In a specific embodiment of the invention, data characterizing fluid flow and suction may be displayed.

In other various embodiments of the invention, the template 500 provides surgical guidance. Such guidance may be preprogrammed and/or interpretive. For example, processor 108 may include a collection of selectable videos and/or still images, with each video and/or still image pertaining, without limitation, to an anatomy of healthy or diseased tissue (such as, for example, lesions of the eye), a particular surgical procedure and/or a complication that may arise during surgery. The video(s) may be produced by experienced surgeons. The selected video 509 and/or still image may be displayed in the template 500. If a certain step in an operation needs to be reviewed or if a complication is encountered, the surgeon or other observer can temporarily pause the surgery being performed and play the appropriate portion of video 509 so as to obtain substantially instant expert advice. In other embodiments, the video 509 may be played simultaneously with the on-going surgery, with, for example, the surgeon stepping through the surgery based on instructions provided by the video. The video(s) and/or still images may be stored, without limitation, on tape, DVD, or other suitable recording mediums known in the art. Both graphical material and/or audio material may be presented. Operator interface 209 (see FIG. 2) may provide the capability to quickly play an operator selectable portion of the video 509, such as by accelerating the video backwards or forwards, or jumping to a certain video image(s). Portions of the video signal representative of the stereoscopic view pair and/or the template may also be recorded on the storage medium, for playback during surgery or at a later time. Processor 8 may freeze an image of a particular subject on a display, allowing, for example, the frozen image to be compared with another image that is being displayed in real time.

In other embodiments of the invention, a plurality of images of an eye may be presented on the display monitor 112 simultaneously. One or more of these images may be retrieved from a storage device and displayed, for example. One or more of these images may be captured by video cameras positioned with various angles and placements with respect to the eye.

Other guidance provided by the template 500 may include, without limitation, medical diagnostic advice based on inputs from the surgeon and/or from information that processor 8 obtains directly from the stereoscopic view pair. For example, a surgeon may provide to the processor 8, via the operator interface 209, symptoms, key words, and/or questions. Processor's 8 responses to the query may involve various levels of search capability, artificial intelligence, interpretive capability, and/or pattern recognition capability, as known in the art. Using pattern recognition, the processor 8 may, for example, identify various structures and/or diseased parts. Alarms based on an occurrence of a triggering event may be provided. Such alarms may be graphical and/or audio.

Processor 8 may be connected to a network, which may be the Internet. Thus, for example, queries of the surgeon and/or various portions of the video signal representative of a stereoscopic view pair and/or template may be transmitted to various remote locations. Observers and/or processors at the remote location(s) may provide, without limitation, advice, diagnosis, tutorial, pictorial, audio, or video information to processor 8 that can then be presented to the surgeon and/or stored in memory. Communication via the network may occur in real-time, with minimal delay.

Various embodiments of the invention may be implemented as a computer program product for use with a computer system. Such implementation may include a series of computer instructions fixed either on a tangible medium, such as a computer readable media (e.g., a diskette, CD-ROM, ROM, or fixed disk), or fixed in a computer data signal embodied in a carrier wave that is transmittable to a computer system via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web).

The present invention may be embodied in other specific forms without departing from the true scope of the invention, as defined by the appended claims. The described embodiments are to be considered in all respects only as illustrative and not restrictive.

1. A method of providing visual documentation for an ophthalmic surgical procedure comprising:
providing at least one video camera, at least one monitor and a processor, the processor in signal communication with the at least one video camera and the at least one monitor;
capturing an image of a first eye using the video camera;
selecting a first template having a graphical content pertinent to the surgical procedure; and
displaying the first template overlaid contemporaneously with the image of the first eye on the monitor.

2. A method according to claim 1 further including:
reducing reflections in the captured image of the first eye.

3. A method according to claim 2, wherein reducing reflections includes illuminating the first eye by sequencing a plurality of light sources on and off.

4. A method according to claim 2, wherein reducing reflections includes illuminating the first eye from a plurality of angles.

5. A method according to claim 2, wherein reducing reflections includes:
capturing a sequence of images of the first eye from a plurality of video cameras.

6. A method according to claim 4, further including:
processing the captured sequence of images of the first eye to form the image of the first eye such that at least one reflection is substantially removed from the sequence of images.

7. A method according to any of claims 2-6 wherein displaying the image of the first eye includes displaying a stereoscopic image.

8. A method according to claim 1, wherein capturing the image includes using a wide dynamic range system to reduce the saturation of highlights and thereby to reduce the effects of glare.

9. A method according to claim 8, wherein the wide dynamic range is achieved using a video camera with adjustable sensitivity and using corresponding scaling techniques.

10. A method according to claim 8, wherein the wide dynamic range is achieved by illuminating the first eye with a variable brightness light source.

11. A method according to claim 8, further comprising:
illuminating the first eye with a variable brightness light source; and
adjusting the brightness of the variable brightness e light source using the image of the first eye captured with the video camera and a feedback loop, thereby reducing glare.

12. A method according to any of claims 8-11 wherein displaying the image of the first eye includes displaying a stereoscopic image.

13. A method according to claim 1, further comprising:
processing the captured image of the first eye to change at least one of the resolution, contrast, brightness, magnification and color of the image.

14. A method according to claim 1, further comprising:
processing the captured image of the first eye to enhance an edge in the image.

15. A method according to claim 1, further comprising:
processing the captured image of the first eye such that a first portion of the image is displayed at a higher magnification than a second portion of the image.

16. A method according to claim 1 wherein a central portion of the image is displayed at a higher magnification than the rest of the image.

17. A method according to claim 1, further comprising:
illuminating the first eye with a laser such that a user is not exposed to laser light.

18. A method according to any of claims 16-17, wherein displaying the image of the first eye includes displaying a stereoscopic image.

19. A method according to claim 1, further comprising:
selecting a wavelength of light; and
illuminating the first eye with light at the selected wavelength to enhance display of a given structure of the first eye.

20. A method according to claim 19 wherein selecting a wavelength of light includes selecting a range of wavelengths and illuminating the first eye includes illuminating the first eye over the range of wavelengths.

21. A method according to claim 1, further comprising
processing the captured image of the first eye such that only a given range of wavelengths of light are displayed on the monitor.

22. A method according to claim 1, wherein capturing the image of the first eye includes capturing the image at a selected set of wavelengths.

23. A method according to any of claims 19-22 wherein displaying the image of the first eye includes displaying a stereoscopic image.

24. A method according to claim 1 further comprising:
identifying two points in the image of the first eye;
measuring the distance between the points; and
displaying the distance measurement on the monitor.

25. A method according to claim 24, wherein the identified points correspond to one of pupil size, lesion size, incision length and corneal curvature.

26. A method according to claim 1, wherein the first template shows a position and extent of an incision.

27. A method according to claim 26, wherein the first template includes a circle, the circle positioned and sized to correspond to the incision for a capsularhexis procedure.

28. A method according to claim 26 wherein the first template shows the position and extent of the incision for one of cataract removal and corneal repair.

29. A method according to claim 1, wherein the first template shows a first measurement of the major axes of astigmatism of the cornea and the optical center of the cornea.

30. A method according to claim 29 further comprising:
displaying a second template showing a second measurement of the major axes of astigmatism and the optical center of the cornea overlaid contemporaneously with the image of the first eye on the monitor, wherein the second measurement is made subsequent to the first measurement.

31. A method according to any of claims 26-30 wherein displaying the image of the first eye includes displaying a stereoscopic image.

32. A method according to claim 1 wherein the first template includes a suturing pattern.

33. A method according to claim 1 wherein the first template includes an indication of a position and an orientation for a surgical instrument.

34. A method according to claim 32-33 wherein displaying the image of the first eye includes displaying a stereoscopic image.

35. A method according to claim 1 for displaying visual documentation for implanting an implant in the first eye, further comprising:
selecting an area of the first eye for placement of the implant wherein the first template identifies the selected area for the implant.

36. A method according to claim 35 wherein the implant is a conjunctival tissue transplant, further comprising:
displaying a second template on the first eye, the second template identifying the conjunctival tissue transplant to be harvested for transplant.

37. A method according to claim 35 for performing corneal transplant surgery on the first eye wherein the implant is a corneal transplant, further comprising:
placing the corneal transplant onto the first eye; and
displaying a second template on the first eye, the second template identifying a suture pattern for the transplant.

38. A method according to claim 1 wherein the first template includes a circle centered at the center of the cornea for identifying a corneal transplant tissue to be removed from the first eye.

39. A method according to claim 1 wherein the first template shows a location of a portion of an iris of the first eye, the portion to be removed.

40. A method according to any of claims 35-39 wherein displaying the image of the first eye includes displaying a stereoscopic image.

41. A method according to claim 1 further comprising:
displaying instructions on the monitor for performing a procedure.

42. A method according to claim 1 further comprising:
displaying a video of a surgeon performing a procedure on the monitor.

43. A method according to claim 41 wherein the video of the surgeon is displayed in a separate window on the monitor.

44. A method according to claim 1 further including:
displaying an additional image of the first eye on the monitor.

45. A method according to claim 44 wherein displaying an additional image of the first eye includes retrieving the additional image from a storage medium.

46. A method according to claim 1 further comprising:
receiving an input identifying an area of the image of the first eye; and
displaying the identified area on the monitor.

47. A method according to claim 1 further comprising:
receiving real-time data related to a surgical procedure; and
displaying the real-time data on the monitor.

48. A method according to claim 47 wherein the real-time data includes one of a running time of the procedure, a time of day, a blood pressure, a pulse and an oxygen level.

49. A method according to claim 47 wherein the real-time data includes at least one of a variable characterizing fluid flow and a variable characterizing suction.

50. A method according to claim 47 wherein the real-time data includes a variable characterizing the first eye, the variable chosen from the group consisting of an intraocular pressure, a temperature, a refractive power, a wave front map, a topography, an area of a tissue, a volume of a tissue, and a corneal thickness.
